(19) United States
(12) Patent Application Publication
(10) Pub. No.: US 2011/0124891 A1
(43) Pub. Date: May 26, 2011

(54) ALPHA-PENTAFLUOROSULFANYL ALDEHYDES, KETONES AND ACIDS

(76) Inventors: John Welch, Albany, NY (US); Silvana Ngo, Albany, NY (US); Dong Sung Lim, Rochelle Park, NJ (US)

(21) Appl. No.: 12/673,547

(22) PCT Filed: Aug. 16, 2008
(86) PCT No.: PCT/US08/73408

§ 371 Date: Feb. 11, 2011

Related U.S. Application Data
(60) Provisional application No. 60/956,154, filed on Aug. 16, 2007.

(51) Int. Cl. C07C 381/00; C07D 303/48; C07D 301/02

(52) U.S. Cl. 549/519; 568/74; 560/147; 562/426; 562/605; 549/549

(57) ABSTRACT

Compounds of formula (I): are disclosed. In these compounds Y is —CH(OH)—, —CH(NHR⁶)—, —C(=0)-, —CH═CHCO— or—formula (II)—and R¹ and R² are hydrogen, OH, alkyl, alkoxy, benzyloxy and aryl, and, when Y is —CH(OH)—, additionally alkenyl or alkynyl. Processes for the production of these compounds are also disclosed.

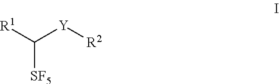

### CROSS REFERENCE TO RELATED APPLICATIONS

This application claims the priority of U.S. provisional application 60/956,154, filed Aug. 16, 2007, the entire contents of which are incorporated herein by reference.

### FIELD OF THE INVENTION

The invention relates to alpha-pentafluorosulfanyl aldehydes and ketones, their preparation and some further reactions utilizing them to make, among other things, esters and acids.

### BACKGROUND OF THE INVENTION

The development of electron-beam resist systems used for photomask fabrication has been focused on improving the sensitivity, resolution, and etch resistance of the resist materials. Among the first resists used for this application were members of a family of positive-tone resists that undergo chain scission. Chain-scission resists operate on the basis of a radiation-induced reduction in the molecular weight of the comprising polymer; this reduced molecular weight results in a solubility differential in the appropriate developing solution. The first and classic example of a chain-scission resist for e-beam applications is poly(methylmethacrylate) or PMMA. This simple resist material has been shown to provide resolution that is among the highest for any resist for any lithographic application. It has been the touchstone in the development of all e-beam-sensitive materials since its initial use in the late 1960s. Numerous publications have reported on the optimization of PMMA-based resists. Of particular note is the incorporation of highly electron-withdrawing groups, such as halogens, at the α-position of the acrylate moiety. In response to the limitations of earlier resists, a new chain-scission positive-tone resist was developed in the mid-1990s that was based upon poly(methyl-m-chloroacrylate-co-α-methylstyrene). This new resist, ZEP from Nippon Zeon, has found wide acceptance and is currently being used in 180-nm device design rule mask production at doses of ˜8 μC/cm² on 10-kV exposure systems. The implementation of a dry Cl₂/O₂ etch process with high etch anisotropy allowed for high-fidelity image transfer of mask images with dimensions as small as 250 nm. However, despite the widespread acceptance and use of ZEP, it does not fully satisfy all of the industry's current and future requirements. Some desirable enhancements as the industry begins to migrate toward higher-voltage exposure systems include improved contrast (>2), enhanced RIE resistance (>2:1 resist/Cr etch ratio), and improved sensitivity (<8 μC/cm² at 10 kV or <25 μC/cm² at 50 kV). Because of the large electron-beam cross section associated with the SF₅ group and subsequent facile decomposition reactions, polymers in which the chlorine of ZEP has been replaced by pentafluorosulfan are expected to offer improved resolution.

### SUMMARY OF THE INVENTION

There is provided, in accordance with an embodiment of the invention, a compound of formula I:

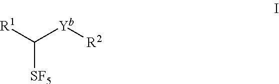

wherein
Y^{b} is chosen from the group consisting of —CH(OH)—, —CH(NHR⁶)—, —CH═CHCO— and

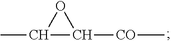

R¹ is chosen from:
   H,
   arylalkyl,
   alkyl,
   alkyl wherein up to three H atoms are replaced with halogen, acyl, alkoxyalkyl, heteroaryl, loweralkoxy, haloalkoxy, aryl, phenoxy, benzyloxy; and arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, benzyloxy; and
R² is chosen from H, OH, alkyl, alkoxy and aryl, and, when Y is —CH(OH)—, additionally alkenyl or alkynyl; and
R⁶ is chosen from optionally substituted alkyl and optionally substituted phenyl.

In another embodiment of the invention there is provided a compound of formula If:

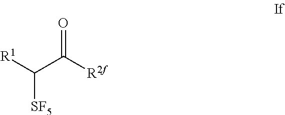

wherein R^{2f} is chosen from H, OH, benzyloxy, aryl, alkyl other than methyl and alkoxy other than methoxy.

In another embodiment of the invention there is provided a compound of formula Vaa:

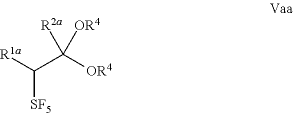

wherein
R^{1a} is chosen from:
   arylalkyl,
   alkyl,
   alkyl wherein up to three H atoms are replaced with halogen, acyl, alkoxyalkyl, heteroaryl, loweralkoxy, haloalkoxy, aryl, phenoxy, benzyloxy; and
   arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, benzyloxy;
R^{2a} is H, alkyl or aryl; and
each R⁴ is independently C₁-C₄ alkyl.

There are also provided, in accordance with some embodiments of the invention, processes for preparing a compound of formula I. One process relates to preparing a compound of formula Ia:

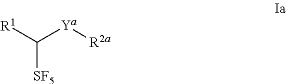

wherein
Y^{a} is chosen from —CH(OH)— and —CH(NHR⁶)—;
R is optionally substituted alkyl or optionally substituted phenyl;
R¹ is H, aralkyl or alkyl; and
R^{2a} is H, alkyl or aryl.

The process comprises:
(1) providing a compound of formula IIa:

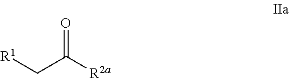

wherein R¹ and R^{2a} are as defined above;
(2) converting the compound of formula IIa to a compound of formula IIIa:

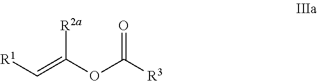

wherein R³ is C₁-C₈ alkyl;
(3) converting the compound of formula IIIa to a compound of formula IVa:

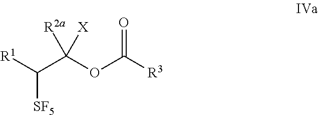

wherein X is Cl or Br;
(4) converting the compound of formula IVa to a compound of formula Va:

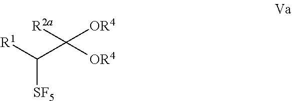

wherein each R⁴ is independently C₁-C₄ alkyl;
(5) converting the compound of formula Va to a compound of formula Ia in which Y^{a} is —C(═O)—; and,
(6) converting the compound of formula Ia in which Y^{a} is —C(═O)— to a compound of formula Ia in which Y^{a} is —CH(OH)— or —CH(NHR⁶)—.

Another process relates to preparing a compound of formula Ib:

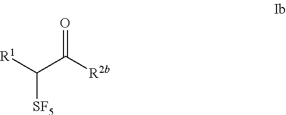

wherein R^{2b} is chosen from OH, alkoxy and benzyloxy.

Another process relates to preparing a compound of formula Id:

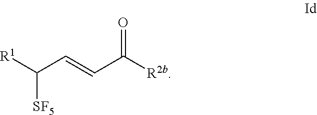

Another process relates to preparing a compound of formula Ie:

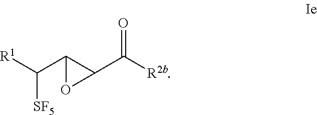

### DETAILED DESCRIPTION OF THE INVENTION

Compounds of formula I are provided

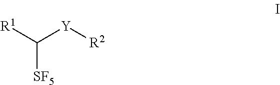

In these compounds Y is —CH(OH)—, —CH(NHR⁶)—, —C(═O)—, —CH═CHCO— or

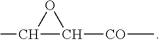

R¹ may be chosen from: H, arylalkyl, alkyl, substituted alkyl and substituted arylalkyl. When alkyl is substituted, up to three H atoms may be replaced with halogen, acyl, alkoxyalkyl, heteroaryl, loweralkoxy, haloalkoxy, aryl, phenoxy and/or benzyloxy. When arylalkyl is substituted, up to three H atoms on the aryl may be replaced with halogen, haloalkyl, alkyl, acyl, alkoxyalkyl, heteroaryl, loweralkoxy, haloalkoxy, cyano, aryl, benzyl, phenoxy and/or benzyloxy. R² may be H, OH, alkyl, alkoxy or aryl.

In some embodiments of the invention, R¹ is n-pentyl and R² is H. In some embodiments, R¹ is n-propyl and R² is H. In some embodiments R¹ is benzyl and R² is H. In some embodiments R¹ and R² are both H. In some embodiments R¹ is H and R² is phenyl. In some embodiments, X is Br. In other embodiments, X is Cl. In some embodiments, Y is —(C═O)—. In other embodiments, Y is —CH(OH)—. In some embodiments, when Y is —CH(OH)—, R² is methyl, vinyl or ethynyl. In other embodiments, Y is —CH(NHR⁶)—. In some embodiments, R⁶ is 4-methoxyphenylmethyl. In some embodiments, Y is —CH═CHCO—. In other embodiments, Y is

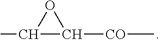

In some embodiments, when Y is —CH═CHCO—

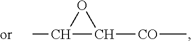

R² is OH or C₁-C₆ alkoxy.

For use in the preparation of electron beam resists, compounds of formula Ib:

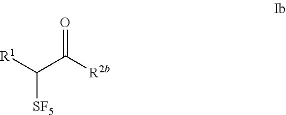

wherein R^{2b} is OH or alkoxy, may be incorporated into polymers analogous to ZEP by processes well known to persons of skill in the art. In these polymers α-chloromethacrylate is replaced by Ib.

Analogously to the trifluormethylated amines and amino alcohols that have been shown to have utility as ligands or chiral auxiliaries [see, for example, Andres et al., Eur. J. Org. Chem. 2004(7), 1558-1566; Katagiri et al., Tetrahedron: Asymmetry 2006 17(8), 1157-1160; and Katagiri et al., J. Fluor. Chem., 2005, 126(8): 1134-1139], it is expected that the present compounds of formula I in which Y is —CH(OH)— or —CH(NHR)— may be useful as ligands or chiral auxiliaries and may exhibit superior properties in this regard because of the larger pentafluorosulfan group. Other possible uses for compounds of formula I (including those in which Y is —C(═O)—) are as biodegradable water repellent coatings, which can be useful in treatment of fibers and paper; coatings for the treatment of ceramics and stone to resist water permeability; as agents for electronics cleaners, due to the hydrophobicity of the SF₅ group; as biodegradable volatile solvents for the dispersal of paints; in the preparation of compounds which may be used as ligands in chemical vapor deposition processes; in the preparation of benzimidazoles for antineoplastic therapy; in the preparation of quinazoline derivatives for treating hypertension and myocardial infarction; in the preparation of benzodiazepin-2-ones as HIV reverse transcriptase inhibitors; in the preparation of an analogue of efavirenz with an SF₅-methylene side chain. Compounds of formula I in which Y is CH(OH) may also be useful as ligands for catalysts; in preparing compounds for use as liquid crystals; as a side chain constituent in anti-depressant analogues of befloxatone; in the preparation of metalloprotease inhibitors; and in the preparation of glucocorticoid receptor inhibitors. The SF₅ amines may be used as chiral ligands for asymmetric catalysis, e.g. nucleophilic additions to carbonyls, Simmons-Smith cyclopropanation; and as amphoteric non-ionic surfactants.

In some embodiments, the compound of formula I is selected from one of the following:

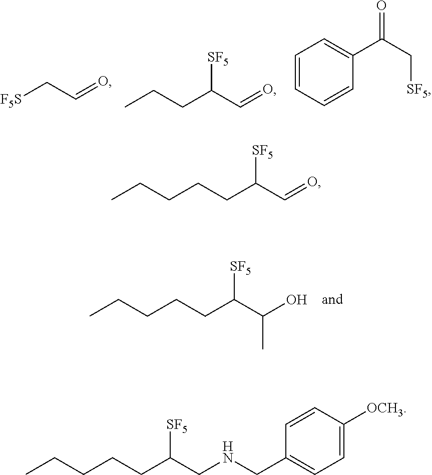

Processes for preparing compounds of the invention generally pass through a ketone or aldehyde intermediate Iaa:

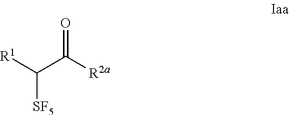

in which R^{2a} is H, alkyl or aryl. The general process comprises the steps of:
(1) providing a compound of formula IIa:

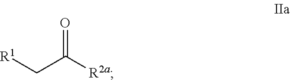

(2) converting the compound of formula Ha to an enol ester of formula IIIa:

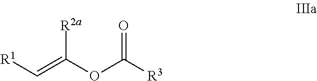

wherein R³ is C₁-C₈ alkyl;
(3) adding the elements of SF₅X across the double bond of IIIa to provide a compound of formula IVa:

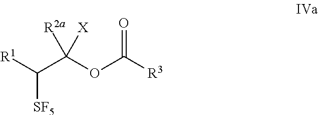

wherein X is Cl or Br;
(4) converting the compound of formula IVa to a ketal of formula Va:

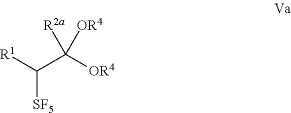

wherein each R⁴ is independently C₁-C₄ alkyl; and
(5) converting the compound of formula Va to a compound of formula Iaa.

Generally the conversion of the ketone or aldehyde IIa to the enol ester IIIa is accomplished by either (a) reacting the compound of formula IIa with an ester of R³COOH in the presence of an acid; or (b) reacting the compound of formula IIa with an anhydride of R³COOH in the presence of a salt of R³COOH. In some embodiments R³ is phenyl or C₁-C₄ alkyl. When the first route is chosen, the ester of R³COOH may be an isoprenyl ester, R³COOCH₂CH₂C(CH₃)═CH₂, and the acid may be a sulfonic acid, particularly toluenesulfonic acid, benzenesulfonic acid or methanesulfonic acid. When the second route is chosen, the anhydride of R³COOH is (R³CO)₂O, conveniently acetic anhydride, and the salt of R³COOH is an alkali metal salt, such as a sodium, potassium or cesium salt.

The addition of SF₅X across the double bond of the enol ester IIIa to provide the pentafluorosulfan IVa may be accomplished by adding a solution of SF₅X, wherein X is Cl or Br, in an alkane or mixture of alkanes. The optimal solvent for SF₅X appears to be a hexane or a mixture of alkanes that is rich in hexanes. In one embodiment, the hexane may be cyclohexane. The reaction may be accelerated by the addition of a radical initiator, such as triethyl borane.

The conversion of the haloester IVa to the ketone or aldehyde Iaa appears to proceed more readily via the acetal than by direct hydrolysis in many cases. The haloester IVa may be treated with an alcohol, such as methanol or ethanol, to form the acetal Va, which can then be hydrolyzed with aqueous acid.

In some embodiments of the invention, the compound of formula Va is one of the following:

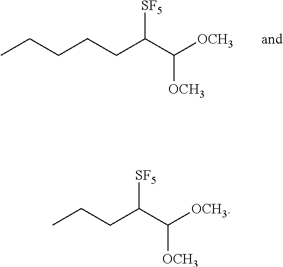

Compounds of formula Ib:

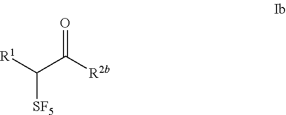

in which R^{2b} is OH or alkoxy may be prepared from compounds of formula Ic:

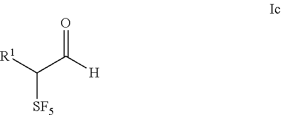

described above by oxidizing the aldehyde to the acid (formula Ib wherein R^{2b} is OH). The oxidation may employ a standard reagent in the art for oxidizing alcohols to acids such as potassium permanganate. If the ester (formula Ib wherein R^{2b} is alkoxy) is desired, the acid 1c may be esterified by procedures well known to persons of skill in the art.

Compounds of formula Id:

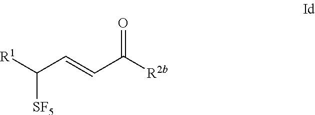

in which R^{2b} is alkoxy, usually C₁-C₄ alkoxy, or benzyloxy may be prepared from the aldehyde Ic:

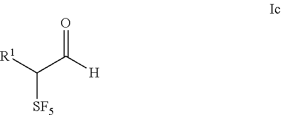

by treatment of Ic with a phosphonate carbanion (Horner-Emmons reaction) or a phosphonium ylide (Wittig). If the free acid is desired (R^{2b}=OH), the ester may be cleaved by any of the methods well known in the art, such as base hydrolysis when R^{2b} is C₁-C₄ alkoxy, and hydrogenolysis when R^{2b} is benzyloxy.

Compounds of formula Ie:

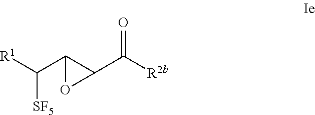

may be prepared by converting a compound of formula Ic to a compound of formula, for example by treatment of Ic with a sulfonium ylide,

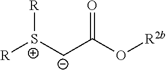

in which R represents any of the common alkyls (usually methyl) employed for sulfonium ylide reactions.

The ketone or aldehyde compound Iaa:

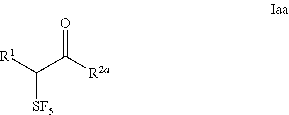

may be converted to a compound of formula I in which Y is —CH(OH)— or —CH(NHR⁶)—. In some embodiments, when R² is H in the compound of formula I in which Y is —C(═O)—, R² in the compound of formula I in which Y is —CH(OH)— is an alkyl group. In some embodiments, the process is practiced without isolation of one or more of the compounds of formula III, IV and V. In some embodiments, the process is practiced isolating the compound of formula IV. In some embodiments, the process is practiced without isolating the compound of formula V. In some embodiments, R³ is methyl. In some embodiments, R⁴ in each instance is methyl.

### DEFINITIONS

Throughout this specification the terms and substituents retain their definitions.

Alkyl is intended to include linear, branched, or cyclic hydrocarbon structures and combinations thereof. When not otherwise restricted, the term refers to alkyl of 20 or fewer carbons. Lower alkyl refers to alkyl groups of 1, 2, 3, 4, 5 and 6 carbon atoms. Examples of lower alkyl groups include methyl, ethyl, propyl, isopropyl, butyl, s- and t-butyl and the like. Cycloalkyl is a subset of alkyl and includes cyclic hydrocarbon groups of 3, 4, 5, 6, 7, and 8 carbon atoms. Examples of cycloalkyl groups include c-propyl, c-butyl, c-pentyl, norbornyl, adamantyl and the like.

C₁ to C₂₀ Hydrocarbon includes alkyl, cycloalkyl, polycycloalkyl, alkenyl, alkynyl, aryl and combinations thereof. Examples include benzyl, phenethyl, cyclohexylmethyl, camphoryl and naphthylethyl. Hydrocarbon refers to any substituent comprised of hydrogen and carbon as the only elemental constituents. The term “carbocycle” is intended to include ring systems in which the ring atoms are all carbon but of any oxidation state. Thus (C₃-C₁₀) carbocycle refers to such systems as cyclopropane, benzene and cyclohexene; (C₈-C₁₂) carbopolycycle refers to such systems as norbornane, decalin, indane and naphthalene. Carbocycle, not otherwise limited, refers to monocycles, bicycles and polycycles.

Alkoxy or alkoxyl refers to groups of 1, 2, 3, 4, 5, 6, 7 or 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. For the purposes of the present patent application alkoxy also includes methylenedioxy and ethylenedioxy in which each oxygen atom is bonded to the atom, chain or ring from which the methylenedioxy or ethylenedioxy group is pendant so as to form a ring. Thus, for example, phenyl substituted by alkoxy may be, for example,

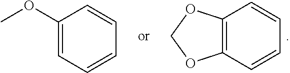

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds). Similarly, thiaalkyl and azaalkyl refer to alkyl residues in which one or more carbons have been replaced by sulfur or nitrogen, respectively. Examples include ethylaminoethyl and methylthiopropyl.

Acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include formyl, acetyl, propionyl, isobutyryl, t-butoxycarbonyl, benzoyl, benzyloxycarbonyl and the like. Lower-acyl refers to groups containing one to four carbons—including the carbonyl carbon.

Aryl and heteroaryl refer to aromatic or heteroaromatic rings, respectively, as substituents. Heteroaryl contains one, two or three heteroatoms selected from O, N, or S. Both refer to monocyclic 5- or 6-membered aromatic or heteroaromatic rings, bicyclic 9- or 10-membered aromatic or heteroaromatic rings and tricyclic 13- or 14-membered aromatic or heteroaromatic rings. Aromatic 6, 7, 8, 9, 10, 11, 12, 13 and 14-membered carbocyclic rings include, e.g., benzene, naphthalene, indane, tetralin, and fluorene and the 5, 6, 7, 8, 9 and 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl means an alkyl residue attached to an aryl ring. Examples are benzyl, phenethyl and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, alkyl, acyl, alkoxyalkyl, hydroxyloweralkyl, phenyl, heteroaryl, benzenesulfonyl, hydroxy, loweralkoxy, haloalkoxy, carboxy, carboalkoxy (also referred to as alkoxycarbonyl), alkoxycarbonylamino, carboxamido (also referred to as alkylaminocarbonyl), cyano, carbonyl, acetoxy, nitro, amino, alkylamino, dialkylamino, mercapto, alkylthio, sulfoxide, sulfone, sulfonylamino, acylamino, amidino, aryl, benzyl, heterocyclyl, phenoxy, benzyloxy, heteroaryloxy, hydroxyimino, alkoxyimino, oxaalkyl, aminosulfonyl, trityl, amidino, guanidino, ureido, and benzyloxy.

The term “halogen” means fluorine, chlorine, bromine or iodine.

In the characterization of some of the substituents, it is recited that certain substituents may combine to form rings. Unless stated otherwise, it is intended that such rings may exhibit various degrees of unsaturation (from fully saturated to fully unsaturated), may include heteroatoms and may be substituted with lower alkyl or alkoxy.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, iodine and chlorine include ³H, ¹⁴C, ³⁵S, ¹⁸F, ³²P, ³³P, ¹²⁵I and ³⁶Cl, respectively. Compounds that contain those radioisotopes and/or other radioisotopes of other atoms are within the scope of this invention. Radiolabeled compounds described herein and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, and as would be understood by the person of skill in the art, the recitation of “a compound” is intended to include salts, solvates and inclusion complexes of that compound as well as any stereoisomeric form, or a mixture of any such forms of that compound in any ratio. Thus, in accordance with some embodiments of the invention, a compound as described herein, including in the contexts of pharmaceutical compositions, methods of treatment, and compounds per se, is provided as the salt form.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

Terminology related to “protecting”, “deprotecting” and “protected” functionalities may occur throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

The abbreviations Me, Et, Ph, Tf, Ts and Ms represent methyl, ethyl, phenyl, trifluoromethanesulfonyl, toluenesulfonyl and methanesulfonyl respectively. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

In the context of the present application, “hexane” in the statement that SF₅X is condensed or dissolved in hexane refers to any alkane having 6 carbon atoms or a mixture of such alkanes, such as straight- and branched-chain C₆H₁₄, a ring-containing alkane of the formula C₆H₁₂ such as cyclohexane or methylcyclopentane. It also includes mixtures of alkanes that are rich in C₆ hydrocarbons. It is uncommon for commercially available solvents to be a pure, single isomer or indeed even pure C₆ hydrocarbon. Most commercial solvents referred to as “hexanes” are in fact mixtures of hydrocarbons—including some C₅ and some C₇ hydrocarbons—that have a boiling range centered on the boiling point of n-hexane. For the purpose of the present invention, the absence of olefins from the mixture is more important than the proportions of particular alkanes.

In general, compounds of formula I may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

Processes for obtaining compounds of formula I are presented below. Other compounds of formula I may be prepared in analogous fashion to those whose synthesis is exemplified herein. The procedures below illustrate such methods. Furthermore, although the syntheses depicted herein may result in the preparation of enantiomers having a particular stereochemistry, included within the scope of the present invention are compounds of formula I in any stereoisomeric form, and preparation of compounds of formula I in stereoisomeric forms other than those depicted herein would be obvious to one of ordinary skill in the chemical arts based on the procedures presented herein.

### Synthetic Methods—Preparation of α-Pentafluorosulfanyl Aldehydes and Ketones

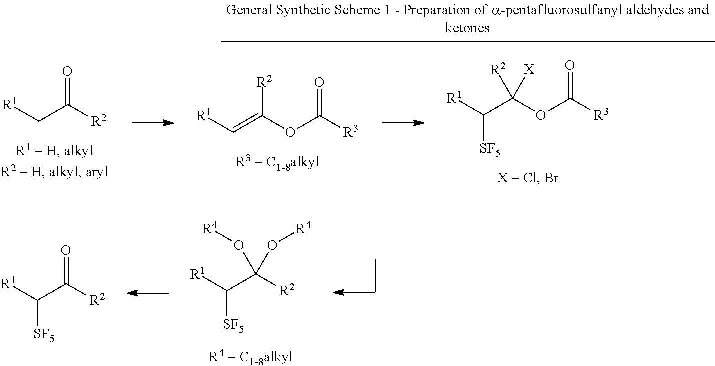

The conversion of the starting ketone or aldehyde to the corresponding enol-ester may be accomplished, for example, by refluxing in isoprenyl acetate and p-TsOH, or by reaction with e.g. potassium acetate in acetic anhydride. The halogenation-pentafluorosulfanation may be accomplished, e.g. by reaction with SF₅X in an inert solvent such as hexane. Conversion of the pentafluorosulfanated compound to the dialkoxy compound may be accomplished, for example, by reaction with an appropriate alcohol. Hydrolysis to the perfluorosulfanated ketone or aldehyde may be achieved, for example, by contacting the dialkoxy compound with aqueous acid, e.g. by refluxing in aqueous HCl or aqueous acetic acid.

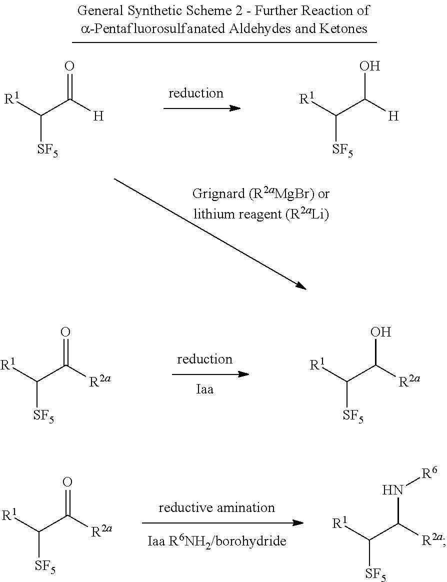

The SF_{S}-aldehydes and ketones may be reduced to the corresponding alcohols, as is known in the art. In the case of the aldehydes, these may also be reduced to alcohols with simultaneous lengthening of the carbon chain, for example by Grignard reaction or reaction of the aldehyde with alkyl lithium in an inert solvent, followed by work-up to yield the alcohol. Similarly, reductive amination of the aldehyde, e.g. by reaction of the aldehyde with a primary amine in the presence of an appropriate borohydride reducing agent and a suitable solvent, produces the subgenus in which Y is —CH(NHR)—.

Preparation of stock solutions of SF₅Cl and SF₅Br in hexane

Into 40 mL of distilled, dried hexane was condensed 10.0 g-13.2 g of SF₅Br with suitable care taken to protect the system from air and moisture. Several repetitions of this procedure resulted in solutions of 1.2-1.6 M. It was found that such solutions could be stored at 4° C. under a nitrogen atmosphere for many months without degradation of the SF₅Br. The solutions were easily transferred for use in the reactions described below by using standard syringe techniques.

Similarly, for SF₅Cl, into 30 mL of distilled dried hexane was condensed 3.3 g-3.7 g of SF₅Cl with suitable care taken to protect the system from air and moisture. Several repetitions of this procedure resulted in solutions of 0.68-0.75 M. It was found that such solutions could be stored at −20° C. under a nitrogen atmosphere for many months without degradation of the SF₅Cl. Such cold solutions were employed in the reactions described below, utilizing standard syringe techniques.

Preparation of enol-acetate of heptaldehyde (hept-1-enyl acetate)

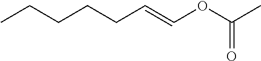

Into a round bottom flask were added heptaldehyde (10 mL, 68 mmol), isopropenyl acetate (38 mL, 342 mmol, 5.0 eq) and p-toluenesulfonic acid (2.6 g, 13.5 mmol, 0.2 eq). The mixture was refluxed overnight. After volatiles were removed in vacuo, the mixture was diluted with Et₂O, washed with water (3×) and brine. The organic layer was dried (MgSO₄), filtered and then concentrated. Distillation of the concentrates at 20 mm Hg yielded the fractions between 75-90° C. as a mixture of (E,Z)-hept-1-enyl acetate.

Alternatively, into a round bottom flask were added heptaldehye (5 mL, 34 mmol), acetic anhydride (7.6 mL, 79 mmol, 2.3 eq) and potassium acetate (0.56 g, 5.7 mmol, 0.17 eq). The mixture was refluxed for 3 hrs. Et₂O was added and the mixture washed with water (2×) and then 20% Na₂CO₃. The organic fraction was washed with brine, dried (Na₂SO₄), filtered and concentrated. Distillation of the concentrates at 20 mm Hg yielded the fractions between 75-90° C. as a mixture of (E,Z)-hept-1-enyl acetate.

Preparation of 1-bromo-2-pentafluorosulfanylheptyl acetate

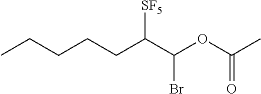

Into a round bottom flask cooled to 0° C. were added a stock solution of SF₅Br in hexane (6.0 mL, 1.6 M, 9.5 mmol, 1.2 eq; stock solution was stored at 4° C. prior to use) and triethyl borane (0.80 mL, 1 M, 0.80 mmol, 0.1 eq). The hept-1-enyl acetate (1.2 g, 7.69 mmol) dissolved in 1 mL hexane was added dropwise. The mixture was allowed to stir at 0° C. for 30 minutes and then quenched with saturated NaHCO₃ solution. The mixture was then extracted with Et₂O and the organic fractions washed with brine, dried (MgSO₄), filtered and concentrated. Purification by flash column chromatography using hexane/methylene chloride afforded the pure 1-bromo-2-pentafluorosulfanylheptyl acetate (1.19 g, 3.28 mmol, 43%). ¹H: δ 4.16 (m, 1H), 2.27 (m, 1H), 2.10 (s, 3H), 1.73 (s, 1H), 1.54 (s, 1H), 1.36 (m, 6H), 0.91 (t, 7.0 Hz, 3H). ¹³C: δ 167.1 (C═O), 90.7 (Cᵢₚₛₒ, qn, 8.2 Hz), 71.8 (C—Br, qn, 5.5 Hz), 31.3, 27.7, 27.5, 22.2, 20.4, 13.8. ¹⁹F: δ 83.3 (9 peaks, 142 Hz, 1F), 58.8 (d, 143 Hz, 4F).

### Preparation of 2-pentafluorosulfanyl-1,1-dimethoxyheptane

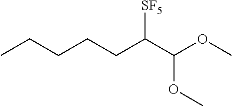

A round bottom flask containing 1-bromo-2-pentafluorosulfanylheptyl acetate (1.19 g, 3.28 mmol) and 2 mL methanol was stirred at room temperature for 3 days. Water was added (1 mL) and then the mixture was extracted with Et₂O. The organic fraction was washed with brine, dried (Na₂SO₄), filtered and concentrated. The crude product contains both the acetal and aldehyde (0.67 g, 2.34 mmol, 71%). ¹H: δ 4.70 (d, 1.5 Hz, 1H), 3.80 (hp, 6.9 Hz, 1H), 3.46 (s, 3H), 3.41 (s, 3H), 1.98 (m, 2H), 1.48 (m, 2.H), 1.29 (m, 4H), 0.86 (m, 3H). ¹³C: δ 104.9 (qn, 5.2 Hz), 88.1 (Cᵢₚₛₒ, qn, 6.4 Hz), 57.1, 56.4, 31.7, 27.5, 26.8 (C_{β}, qn, 3.3 Hz), 22.3, 13.9. ¹⁹F: δ 86.5 (9 peaks, 143 Hz, 1F), 58.5 (dd, 143 Hz, 7.1 Hz, 4F).

### Preparation of α-Pentafluorosulfanylheptaldehyde

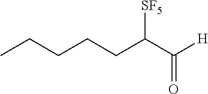

A mixture of 2-pentafluorosulfanyl-1,1-dimethoxyheptane (0.67 g, 2.34 mmol), aqueous HCl (3.5 M, 4 mL) and acetic acid (3 mL) was refluxed for 1 hr. After cooling to room temperature, 10 mL Et₂O was added and the mixture poured into a beaker containing 40 mL of 20% NaHCO₃. The mixture was stirred until no visible bubbling observed and the layers separated. The aqueous layer was extracted with Et₂O and the combined organic fractions dried with Na₂SO₄, filtered and concentrated. NMR of the crude product shows it to be spectroscopically pure (0.46 g, 1.92 mmol, 82%). ¹H: δ 9.59 (m, 1H), 4.24 (m, 1H), 2.13 (m, 2H), 1.29 (m, 6H), 0.86 (t, 3H, 6.9 Hz). ¹³C: δ 190.6 (C═O, qn, 5.1 Hz), 90.4 (Cᵢₚₛₒ, qn, 7.3 Hz), 31.2, 27.2 (C_{β}, 3.6 Hz, qn), 26.1, 22.2, 13.7. ¹⁹F: δ 82.5 (9 peaks, 144 Hz, 1F), 64.1 (dd, 145 Hz, 6.1 Hz, 4F).

### Alkylation of α-pentafluorosulfanylheptaldehyde

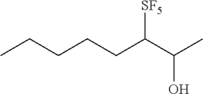

Into a round bottom flask containing MeLi (0.32 mL, 1.6 M, 0.51 mmol, 1.37 eq) in 2 mL Et₂O cooled to −78° C. was added α-pentafluorosulfanylheptaldehyde (0.069 g, 0.288 mmol) dissolved in 0.5 mL Et₂O. The mixture was stirred at −78° C. for 20 minutes and then quenched with saturated NH₄Cl solution. The mixture was then extracted with Et₂O, washed with brine, dried (N₂SO₄), filtered and concentrated. Purification by flash column chromatography using hexane:CH₂Cl₂ afforded the alcohol product as a colorless liquid (0.040 g, 0.156 mmol, 54%). ¹H: δ 4.66 (q, 6.6 Hz, 1H), 3.74 (m, 1H), 1.90 (OH, s, 1H) 1.3-1.22 (m, 8H), 1.26 (d, 6.6 Hz, 3H), 0.89 (5, 6.8 Hz, 3H). ¹³C: δ 95.1 (C—OH, qn, 4.5 Hz), 67.5 (Cᵢₚₛₒ, qn, 4.1 Hz), 31.7, 28.0, 26.8 (qn, 3.3 Hz), 22.3, 21.4, 13.9. ¹⁹F: δ 87.7 (9 peaks, 141 Hz, 1F), 57.4 (td, 141 Hz, 5.3 Hz, 4F).

### Reductive Amination of α-Pentafluorosulfanylheptaldehyde

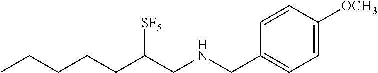

Into a round bottom flask containing a-pentafluorosulfanylheptaldehyde (0.098 g, 0.41 mmol) in 1 mL THF was added p-methoxybenzylamine. The mixture turned cloudy, then cloudy yellow after a few minutes. Sodium triacetoxyborohydride and another mL of THF was added and the mixture cleared into a lemon yellow color after 5 minutes. The mixture was stirred at room temperature for 2 hrs and then quenched with saturated NaHCO₃. The mixture was extracted with Et₂O and the organic fractions washed with brine, dried (MgSO₄), filtered and concentrated. NMR of the crude product shows it to be the imine derivative. Precipitation of the product as a salt was done with ethereal HCl. The product was recrystallized from methanol/ethyl acetate.

### Preparation of enol-acetate of valeraldehyde (pent-1-enyl acetate)

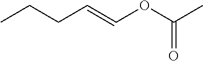

By analogy to the preparation of the enol-acetate of heptaldehyde described above, the enol-acetate of valeraldehyde was prepared.

### Preparation of 1-bromo-2-pentafluorosulfanylpentyl acetate

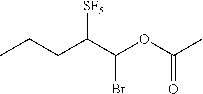

By analogy to the preparation of 1-bromo-2-pentafluorosulfanylheptyl acetate described above, 1-bromo-2-pentafluorosulfanylpentyl acetate was prepared in 74% yield. NMR: ¹H: δ 4.18 (qn, 6.3 Hz, 1H), 2.27 (m, 1H), 2.12 (2, 3H), 1.78 (m, 1H), 1.58 (m, 1H), 1.24 (m, 2H), 1.01 (t, 7.3 Hz, 3H). ¹³C: δ 167.2 (C═O), 90.3 (Cᵢₚₛₒ, qn, 8.0 Hz), 71.8 (C—Br, qn, 5.4 Hz), 29.7, 21.1, 20.6, 13.6. ¹⁹F: δ 83.3 (9 peaks, 143 Hz, 1F), 58.5 (d, 142 Hz).

### Preparation of 2-pentafluorosulfanyl-1,1-dimethoxypentane

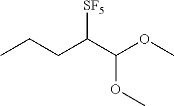

By analogy to the preparation of 2-pentafluorosulfanyl-1,1-dimethoxyheptane described above, 2-pentafluorosulfanyl-1,1-dimethoxypentane was prepared in 62% yield. NMR: ¹H: δ 4.71 (d, 1.5 Hz, 1H), 3.82 (qn, 7.0 Hz, 1H), 3.47 (s, 3H), 3.42 (s, 3H), 1.97 (m, 2H), 1.52 (m, 2H), 0.91 (t, 7.2 Hz, 3H). ¹³C: δ 104.9 (qn, 5.2 Hz), 87.7 (Cᵢₚₛₒ, qn, 6.5 Hz), 57.2, 56.5, 28.9, 21.1, 13.9. ¹⁹F: δ 86.5 (9 peaks, 143 Hz, 1F), 58.5 (dd, 143 Hz, 6.9 Hz, 4F).

### Preparation of α-Pentafluorosulfanypentaldehyde

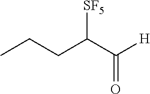

By analogy to the preparation of α-pentafluorosulfanylheptaldehyde described above, α-pentafluorosulfanypentaldehyde was prepared in 24% yield. NMR: ¹H: δ 9.61 (m, 1H), 4.26 (m, 1H), 2.13 (m, 2H), 1.33 (m, 2H), 0.96 (t, 7.3 Hz, 3H). ¹³C: δ 190.6 (C═O, qn, 5.3 Hz), 90.1 (Cᵢₚₛₒ, qn, 7.0 Hz), 29.2 (C_{β}, qn, 3.6 Hz), 19.8, 13.6. 19F: δ 82.6 (9 peaks, 143 Hz, 1F), 64.2 (dd, 144 Hz, 6.3 Hz, 4F).

### Preparation of enol-acetate of acetophenone (1-phenylvinyl acetate)

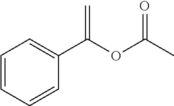

By analogy to the preparation of the enol-acetate of heptaldehyde, the enol-acetate of acetophenone was prepared.

### Preparation of Pentafluorosulfanylacetophenone

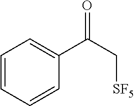

Into a round bottom flask cooled to −30° C. were added 1-phenylvinyl acetate (0.30 g, 1.85 mmol) and a stock solution of SF₅Cl in hexane (5.4 mL, 0.68 M, 3.67 mmol, 2 eq; stock solution was stored at −20° C. prior to use). Triethyl borane (0.56 mL, 1 M, 0.56 mmol, 0.3 eq) was then added dropwise. The mixture was allowed to stir at −30° C. for 1 hr and then quenched with saturated NaHCO₃ solution. The mixture was then extracted with Et₂O and the organic fractions washed with brine, dried (MgSO₄), filtered and concentrated. Purification by flash column chromatography using hexane/methylene chloride afforded the pure pentafluorosulfanylacetophenone in 44% yield. NMR: ¹H: δ 7.99 (d, 8.0 Hz, 2H), 7.63 (t, 7.4 Hz, 1H), 7.51 (t, 7.7 Hz, 2H), 4.86 (qn, 7.8 Hz, 2H). ¹³C: δ 187.0 (C═O, qn, 3.6 Hz), 135.5, 134.5, 129.1, 129.0, 71.6 (Cᵢₚₛₒ, qn, 13.0 Hz). ¹⁹F: δ 80.5 (9 peaks, 145 Hz, 1F), 72.1 (dm, 146 Hz, 4F).

### Preparation of 1-bromo-2-pentafluorosulfanyl-3-phenylpropyl acetate

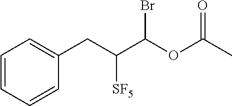

By analogy to the preparation of 1-bromo-2-pentafluorosulfanylheptyl acetate described above, starting from 3-phenylpropanal the title compound was prepared. NMR: ¹H: δ 7.35 (m, 5H), 4.57 (m, 1H), 3.81 (m, 1H), 3.50 (d, 1H, 9.5 Hz), 3.45 (d, 1H, 9.5 Hz), 2.18 (s, 3H). ¹³C: δ 166.9 (C═O), 136.8, 128.7, 128.6, 127.2, 91.9 (Cᵢₚₛₒ, m), 71.6 (C—Br, qn, 5.0 Hz), 33.4, 20.5. ¹⁹F: δ 82.6 (9 peaks, 1F, 144 Hz), 60.5 (d, 4F, 144 Hz).

### Preparation of α-SF5-3-phenylpropionaldehyde

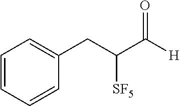

By analogy to the preparation of 1-bromo-2-pentafluorosulfanylheptyl acetate described above, starting from 1-bromo-2-pentafluorosulfanyl-3-phenylpropyl acetate, the title compound was prepared. NMR: 1H, 9.64 (m, 1H), 7.33-7.27 (m, 2H), 7.27-7.23 (m, 1H), 7.19-7.15 (m, 2H), 4.61 (m, 1H), 3.49 (m, 2H). ¹³C, 189.2 (qn, 4.7 Hz), 134.6, 129.2, 129.0, 127.6, 90.2 (Cᵢₚₛₒ, qn, 6.5 Hz), 33.0 (qn, 4.2 Hz). ¹⁹F: 82.0 (9 peaks, 144 Hz, 1F), 65.1 (dd, 144 Hz, 6.2 Hz, 4F).

### Preparation of 1-bromo-2-pentafluorosulfanylethyl acetate

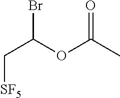

The title compound was prepared, starting from vinyl acetate, analogously to the preparation of 1-bromo-2-pentafluorosulfanylheptyl acetate described above. NMR: ¹H: δ 7.0 (dd, 10.3 Hz, 1.8 Hz, 1H), 4.32 (m, 1H), 4.14 (m, 1H), 2.07 (s, 3H); ¹³C: δ 167.3, 74.2 (Cᵢₚₛₒ, qn, 14.0 Hz), 66.7 (qn, 5.1 Hz), 20.2; ¹⁹F: δ 79.8 (9 peaks, 145 Hz, 1F), 64.9 (td, 146 Hz, 7.8 Hz, 4F).

By analogous procedures the following haloesters were prepared from the corresponding enol esters in the yields shown

| R¹ | R² | reagent | yield |
|---|---|---|---|
| H | C₆H₁₃ | SF₅Cl | 66% |
| Et | n-Pr | SF₅Cl | 15% |
| Et | n-Pr | SF₅Br | 50% |
| H | CH₃ | SF₅Cl | 92% |

### Preparation of 2-pentafluorosulfanyl-1,1-dimethoxyethane

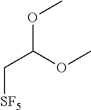

By analogy to the preparation of 2-pentafluorosulfanyl-1,1-dimethoxyheptane described above, the title compound was prepared from 1-bromo-2-pentafluorosulfanylethyl acetate. NMR: ¹H: δ 4.80 (t, 5.1 Hz, 1H), 3.72 (m, 2H), 3.35 (s, 6H); ¹³C: δ 99.9 (qn, 5.3 Hz), 71.6 (Cᵢₚₛₒ, qn, 12.7 Hz), 53.9; ¹⁹F: δ 83.2 (9 peaks, 145 Hz, 1F), 67.0 (td, 145 Hz, 7.9 Hz, 4F). The title compound may be converted to pentafluorosulfanyl acetaldehyde by analogy to procedures described above.

### Alkylation of α-Pentafluorosulfanylaldehyde

Using an alkyllithium:

Into a round bottom flask containing MeLi (0.32 mL, 1.6 M, 0.51 mmol, 1.37 eq) in 2 mL Et₂O cooled to −78° C. was added the α-pentafluorosulfanylheptaldehyde (0.069 g, 0.288 mmol) dissolved in 0.5 mL Et₂O. The mixture was stirred at −78 C for 20° and then quenched with saturated NH₄Cl solution. The mixture was then extracted with Et₂O, washed with brine, dried (Na₂SO₄), filtered and concentrated. Purification by flash column chromatography using hexane:CH₂Cl₂ afforded the alcohol product as a colorless liquid (0.040 g, 0.156 mmol, 54%).

3-Pentafluorosulfanyl-octan-2-ol

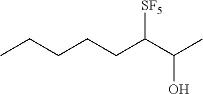

¹H, 4.66 (q, 6.6 Hz, 1H), 3.74 (m, 1H), 2.10 (m, 1H), 1.90 (OH, s, 1H) 1.80 (m, 1H), 1.65 (m, 1H), 1.41 (m, 1H), 1.31 (m, 4H), 1.26 (d, 6.6 Hz, 3H), 0.89 (t, 6.8 Hz, 3H).

¹³C, 95.1 (Cᵢₚₛₒ, qn, 4.5 Hz), 67.5 (C—OH, qn, 4.1 Hz), 31.7, 28.0, 26.8 (qn, 3.3 Hz), 22.3, 21.4, 13.9.

¹⁹F: 87.7 (9 peaks, 141 Hz, 1F), 57.4 (td, 141 Hz, 5.3 Hz, 4F).

### 3-Pentafluorosulfanyl-pentan-2-ol

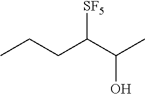

¹H, 4.66 (q, 6.7 Hz, 1H), 3.75 (m, 1H), 2.10 (m, 1H), 1.76 (m, 2H), 1.71 (m, 1H), 1.44 (m, 1H), 1.26 (d, 6.7 Hz, 3H), 0.95 (t, 7.2 Hz, 3H).

¹³C, 94.7 (Cᵢₚₛₒ, qn, 4.6 Hz), 67.4 (C—OH, qn, 4.2 Hz), 28.8 (qn, 3.3 Hz), 21.5, 21.4, 13.9.

¹⁹F: 87.7 (9 peaks, 141 Hz, 1H), 57.4 (td, 141 Hz, 4.7 Hz, 4F).

### 3-Pentafluorosulfanyl-4-phenyl-butan-2-ol

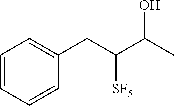

¹H, 7.34-7.27 (m, 2H), 7.26-7.20 (m, 3H), 4.69 (q, 6.8 Hz, 1H), 4.18 (spt, 6.8 Hz, 1H), 3.41 (dd, 15.6 Hz, 6.6 Hz, 1H), 3.28 (dm, 15.6 Hz, 1H), 1.15 (d, 6.8 Hz, 3H).

¹³C: 138.2, 128.8, 128.7, 126.9, 95.6 (qn, 4.2 Hz), 67.3 (qn, 3.5 Hz), 32.7 (qn, 3.9 Hz), 21.6 (bs).

¹⁹F: 86.9 (9 pks, 142 Hz, 1F), 57.6 (dd, 142 Hz, 3.7 Hz, 4F).

Using a Grignard Reagent:

To a solution of vinylmagnesium bromide (0.45 mL, 0.7 M solution in THF, 1.3 eq) in Et₂O (3 mL) at −78° C. was added α-pentafluorosulfanylvaleraldehyde (0.236 mmol). The mixture was stirred at −78° C. for 1 hr, quenched with water and extracted with Et₂O. The organic extracts were washed with brine, dried (MgSO₄) and filtered. Purification via flash column chromatography afforded the pure product in 71% yield.

### 4-Pentafluorosulfanylhep-1-en-3-ol

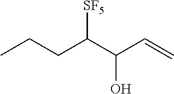

¹H, 5.79 (ddd, 17.1 Hz, 10.5 Hz, 4.5 Hz, 1H), 5.43 (dm, 17.2 Hz, 1H), 5.27 (dm, 10.6 Hz, 1H), 4.99 (bs, 1H), 3.85 (m 1H), 2.07 (m, 1H), 1.76 (m, 1H), 1.60 (m, 1H), 1.40 (m, 1H), 0.91 (t, 7.3 Hz, 3H).

¹³C: 136.8, 117.1, 92.8 (qn, 4.9 Hz), 71.8 (qn, 4.0 Hz), 28.7 (qn, 3.2 Hz), 21.3 (bs), 13.8.

¹⁹F: 87.3 (9 peaks, 142 Hz, 1F), 58.0 (td, 142 Hz, 5.0 Hz, 4F).

Wittig and Horner-Emmons reactions.

A solution of n-BuLi (0.16 mL, 2.0 M, 0.32 mmol) was syringed into a flask containing a solution of triethyl phosphonoacetate (0.064 mL, 0.32 mmol) in Et₂O (3.0 mL) at 0° C. After stirring the mixture at 0° C. for 1 hr, a-SF₅ heptaldehyde (0.0503 g, 0.21 mmol) was added and the mixture further stirred at 0° C. for 2 hrs. The reaction was quenched with water and extracted with Et₂O. The Et₂O layer was washed with water and brine, dried (MgSO₄), filtered and concentrated.

Ethyl 4-pentafluorosulfanylnon-2-enoate (trans:cis=1:0.09)

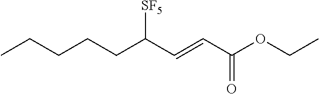

¹H, 6.78 (dd, 15.6 Hz, 10.1 Hz, 1H), 6.0 (d, 15.6 Hz, 1H), 4.31 (m, 1H), 4.20 (qt, 7.1 Hz, 2H), 2.18 (m, 1H), 1.84 (m, 1H), 1.28 (t, 7.1 Hz, 3H), 1.27 (m, 6H), 0.85 (m, 3H).

¹³C, 165.0 (C═O), 140.4 (qn, 3.6 Hz), 127.6 (bs), 87.5 (Cᵢₚₛₒ, qn, 10.7 Hz), 61.0, 31.7 (qn, 3.6 Hz), 31.1, 26.6 (bs), 22.2, 14.1, 13.8.

¹⁹F: 82.8 (9 peaks, 1F), 56.3 (dd, 143 Hz, 5.5 Hz, 4F).

Note: 2 isomers observed in ¹H NMR (1:0.09) but 4 in ¹⁹F NMR (1:0.06:0.05:0.05).

### Ethyl-4-pentafluorosulfanyl-hept-2-enoate

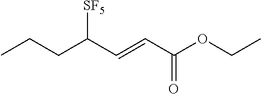

¹H, 6.80 (dd, 15.6 Hz, 10.3 Hz, 1H), 6.01 (d, 15.5 Hz, 1H), 4.34 (m, 1H), 4.22 (q, 7.2 Hz, 2H), 2.18 (m, 1H), 1.88 (m, 1H), 1.32 (m, 1H), 1.30 (t, 7.2 Hz, 3H), 1.19 (m, 1H), 0.93 (t, 7.3 Hz, 3H).

¹³C, 165.0 (C═O), 140.4 (qn, 3.7 Hz), 127.6, 87.2 (Cᵢₚₛₒ, qn, 10.8 Hz), 61.0, 33.7 (qn, 3.5 Hz), 20.2 (bs), 14.1, 13.4.

¹⁹F: 82.8 (9 pks, 143 Hz, 1F), 56.4 (dd, 143 Hz, 5.4 Hz, 4F).

### Ethyl 4-pentafluorosulfanyl-5-phenylpent-2-enoate

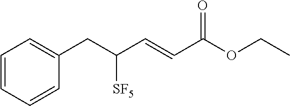

¹H, 7.24 (m, 3H), 7.08 (m, 2H), 6.84 (dd, 15.5 Hz, 10.4 Hz, 1H), 5.66 (d, 15.5 Hz, 1H), 4.54 (m, 1H), 4.13 (qt, 7.2 Hz, 2H), 3.62 (dd, 13.7 Hz, 3.6 Hz, 1H), 3.07 (t, 12.5 Hz, 1H), 1.23 (t, 7.2 Hz, 3H).

¹³C, 164.6 (C═O), 139.2 (qn, 3.6 Hz), 135.4 (bs), 129.2, 128.8, 128.1, 127.3, 88.0 (Cᵢₚₛₒ, qn, 10.2 Hz), 60.8, 38.3 (qn, 4.1 Hz), 14.0.

¹⁹F: 82.4 (9 peaks, 143 Hz), 56.8 (dd, 143 Hz, 5.6 Hz).

### Reduction of α-pentafluorosulfanylaldehyde

To a stirring mixture of the α-pentafluorosulfanylaldehyde (0.30 mmol) and EtOH (1 mL) at 0° C. was added NaBH₄ (1.6 eq). The mixture was stirred at 0° C. for 1 hr and then allowed to warm to RT for another hr. The reaction was quenched with water and then extracted with Et₂O, washed with water, dried (MgSO₄) and filtered. Purification by flash column chromatography using hexane:CH₂Cl₂ afforded the pure product.

### 2-Pentafluorosulfanylpentanol

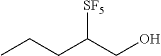

¹H, 4.14 (m, 1H), 3.91 (m, 1H), 3.87 (m, 1H), 2.02 (bq, 7.5 Hz, 2H), 1.95 (bs, 1H), 1.49 (m, 1H), 1.37 (m, 1H), 0.97 (t, 7.4 Hz, 3H).

¹³C, 89.4 (Cᵢₚₛₒ, qn, 6.3 Hz), 61.1 (C—OH, qn, 4.0 Hz), 30.5 (qn, 3.8 Hz), 20.5 (bs), 13.8.

¹⁹F: 86.5 (9 peaks, 142 Hz, 1F), 56.3 (dd, 142 Hz, 5.3 Hz, 4F).

### 2-Pentafluorosulfanylheptanol

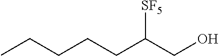

¹H, 4.15 (m, 1H), 3.92 (m, 1H), 3.85 (m, 1H), 2.04 (m, 2H), 1.71 (m, 1H), 1.44 (m, 1H), 1.32 (m, 5H), 0.88 (t, 6.4 Hz, 3H).

¹³C, 89.7 (Cᵢₚₛₒ, m), 61.1 (C—OH, qn, 3.9 Hz), 31.5, 28.4 (qn, 3.7 Hz), 26.9 (bs), 22.3, 13.9.

¹⁹F: 86.5 (9 peaks, 142 Hz, 1F), 56.3 (d, 142 Hz, 4F).

### 2-Pentafluorosulfanyl-3-phenylpropanol

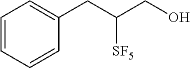

¹H, 7.35-7.29 (m, 2H), 7.28-7.24 (m, 3H), 4.11 (m, 1H), 4.03 (m, 1H), 3.64 (m, 1H), 3.42 (dd, 13.6 Hz, 3.6 Hz, 1H), 3.12 (dd, 14.1 Hz, 12.6 Hz, 1H), 1.75 (bs, 1H). ¹³C:

¹⁹F: 86.0 (9 peaks, 143 Hz, 1F), 56.7 (dm, 143 Hz, 4F).

### Oxidation of α-pentafluorosulfanylaldehyde

To a mixture of the α-pentafluorosulfanylaldehyde (0.30 mmol) in water (1 mL) was added KMnO₄ (1.6 eq). After stirring for 1-2 hrs at RT, satd. NaHSO₃ was added to reduce the excess KMnO₄. The mixture was made basic using solid NaHCO₃ and then filtered through celite. The solution was acidified using concentrated HCl until the product precipitated. The product was extracted using Et₂O or EtOAc. The organic extracts were washed with water, brine, dried (MgSO₄) and filtered. Purification by flash column chromatography afforded the pure product.

### 2-Pentafluorosulfanyl-pentanoic acid

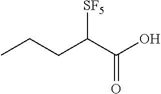

¹H, 9.0 (bs), 4.37 (m, 1H), 2.26 (m, 1H), 2.14 (m, 1H), 1.31 (m, 2H), 0.98 (t, 7.4 Hz, 3H).

¹³C, 170.4 (C═O, qn, 3.1 Hz), 84.8 (Cᵢₚₛₒ, qn, 12.1 Hz), 31.7 (qn, 3.7 Hz), 20.2 (bs), 13.5.

¹⁹F: 80.4 (9 peaks, 145 Hz, 1F), 62.1 (dd, 145 Hz, 5.4 Hz, 4F).

### 2-Pentafluorosulfanylheptanoic acid

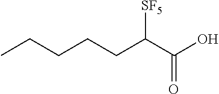

¹⁹F: 80.7 (9 peaks, 145 Hz, 1F), 61.9 (dd, 145 Hz, 4.9 Hz, 4F).

### 2-Pentafluorosulfanyl-3-phenylpropanoic acid

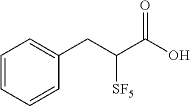

¹H, 7.71 (bs, 1H), 7.32-7.25 (m, 3H), 7.20-7.16 (m, 2H), 4.60 (m, 1H), 3.50 (m, 2H).

¹³C, 169.3 (C═O, qn, 3.0 Hz), 134.5, 129.3, 129.0, 127.8, 85.4 (Cᵢₚₛₒ, qn, 10.8 Hz), 35.6 (qn, 4.2 Hz).

¹⁹F: 80.2 (9 peaks, 146 Hz, 1F), 62.4 (dd, 146 Hz, 5.6 Hz, 4F).

### Reaction with Sulfonium Ylide

To a stirring solution of (2-ethoxy-2-oxoethyl)dimethylsulfonium bromide (0.24 mmol, 1 eq) in 3 mL THF at 0° C. was added n-BuLi (2.0 M, 0.32 mmol, 1.4 eq). The mixture was stirred at 0° C. for 1 hr and then cooled to −78° C. The α-SF5-valeraldehyde (0.233 mmol) was added slowly and the mixture stirred for 2 hrs at −78° C. The reaction was quenched with satd. NH₄Cl and then extracted with Et₂O. The extracts were washed with water, brine, dried (MgSO₄) and then filtered.

Ethyl 3-(1-pentafluorosulfanylbutyl)oxirane-2-carboxylate

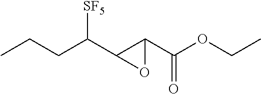

¹H, 4.81 (d, 10.7 Hz, 1H), 4.60 (m, 1H), 4.24 (q, 7.1 Hz, 2H), 3.17 (d, 10.7 Hz, 1H), 1.59 (m, 4H), 1.30 (t, 7.2 Hz, 3H), 0.96 (t, 7.1 Hz, 3H).

¹⁹F: 87.2 (9 peaks, 142 Hz, 1F), 57.9 (dm, 142 Hz, 4F).

Unless otherwise noted, reagents and solvents were used as received from commercial suppliers. NMR coupling constants, J, are reported in Hertz. NMR chemical shifts for proton NMR are reported in ppm (δ) from TMS and were determined relative to residual protons in CDCl₃ (δ=7.24) or C₆D₆ (δ=7.15). NMR chemical shifts for ¹³C NMR are reported in ppm from TMS and were determined relative to the carbon resonance of CDCl₃ (δ=77.00) or C₆D₆ (δ=128.00). ¹⁹F NMR chemical shifts are reported in ppm from the fluorine resonance of CFCl₃ (δ=0) and were determined relative to an external frequency (no internal standard). All ¹H, ¹³C and ¹⁹F NMR spectra were recorded on a Gemini-300 MHz NMR spectrometer at 300, 75.43 and 282.20 MHz respectively and a Brucker 400 MHz NMR spectrometer at 400, 100 and 376 MHz respectively.

The present invention is not limited to the compounds found in the above examples, and many other compounds falling within the scope of the invention may also be prepared using the procedures set forth in the above synthetic schemes. The preparation of additional compounds of formula I using these methods will be apparent to one of ordinary skill in the chemical arts.

The invention has been described in detail with particular reference to some embodiments thereof, but it will be understood by those skilled in the art that variations and modifications can be effected within the spirit and scope of the invention.

1. A compound of formula I: 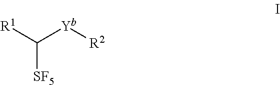
wherein
Y^{b} is chosen from the group consisting of —CH(OH)—, —CH(NHR⁶)—, —CH═CHCO— and 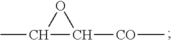
R¹ is chosen from:
H,
arylalkyl,
alkyl,
alkyl wherein up to three H atoms are replaced with halogen, acyl, alkoxyalkyl, heteroaryl, loweralkoxy, haloalkoxy, aryl, phenoxy, or benzyloxy, and
arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, or benzyloxy;
R² is chosen from H, OH, alkyl, alkoxy, benzyloxy and aryl, and, when Y is —CH(OH)—, additionally alkenyl and alkynyl; and
R⁶ is chosen from optionally substituted alkyl and optionally substituted phenyl.

2. A compound according to claim 1 wherein
Y^{b} is chosen from the group consisting of —CH(OH)— and —CH(NHR⁶)—;
R¹ is H, arylalkyl or alkyl; and
R² is H, alkyl or aryl.

3. A compound according to claim 1 wherein R¹ is C₁ to C₆ alkyl.

4. A compound according to claim 1 wherein R¹ is benzyl or substituted benzyl.

5. A compound according to claim 1 wherein R² is H.

6. A compound according to claim 1 wherein R¹ is H and R² is phenyl.

7. A compound according to claim 1 wherein Y^{b} is —CH(OH)—.

8. A compound according to claim 1 wherein Y^{b} is —CH(OH)— and R² is chosen from C₁-C₆ alkyl, alkenyl, alkynyl and aryl.

9. A compound according to claim 1 wherein Y^{b} is —CH(NHR⁶)—.

10. A compound according to claim 1 wherein
Y^{b} is —CH═CHCO—; and
R² is chosen from OH and alkoxy.

11. A compound according to claim 1 wherein Y^{b} is 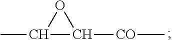
and R² is chosen from OH and alkoxy.

12. A compound of formula If: 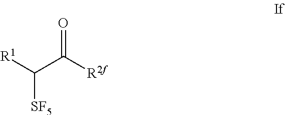
wherein
R¹ is chosen from:
H,
arylalkyl,
alkyl,
alkyl wherein up to three H atoms are replaced with halogen, acyl, alkoxyalkyl, heteroaryl, loweralkoxy, haloalkoxy, aryl, phenoxy, or benzyloxy, and
arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, or benzyloxy;
R^{2f} is chosen from H, OH, benzyloxy, aryl, alkyl other than methyl, and alkoxy other than methoxy.

13. A compound according to claim 12 wherein R^{2f} is H.

14. A compound of formula Vaa: 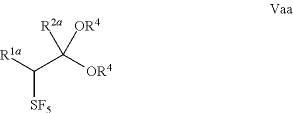
wherein
R^{1a} is chosen from:
arylalkyl,
alkyl,
alkyl wherein up to three H atoms are replaced with halogen, acyl, alkoxyalkyl, heteroaryl, loweralkoxy, haloalkoxy, aryl, phenoxy, or benzyloxy, and
arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, or benzyloxy;
R^{2a} is H, alkyl or aryl; and
each R⁴ is independently C₁-C₄ alkyl.

15. A process for preparing a compound according to claim 1 of formula Ia: 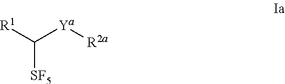
wherein
Y^{a} is —CH(OH)— or —CH(NHR⁶)—;
R¹ is H, arylalkyl or alkyl;
R^{2a} is H, alkyl or aryl; and
R⁶ is chosen from optionally substituted alkyl and optionally substituted phenyl;
comprising:
(1) providing a compound of formula IIa: 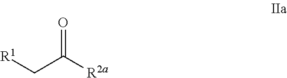
wherein R¹ and R^{2a} are as defined above;
(2) converting the compound of formula IIa to a compound of formula IIIa: 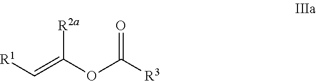
wherein R³ is C₁-C₈ alkyl;
(3) converting the compound of formula Ma to a compound of formula IVa: 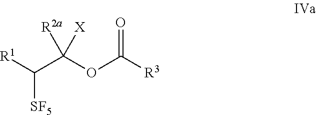
wherein X is Cl or Br;
(4) converting the compound of formula IVa to a compound of formula Va: 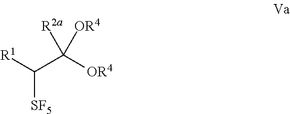
wherein each R⁴ is independently C₁-C₄ alkyl;
(5) converting the compound of formula Va to a compound of formula VIa 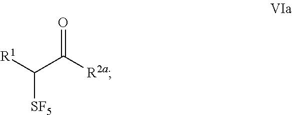
and
(6) converting the compound of formula VIa to a compound of formula Ia in which Y^{a} is —CH(OH)— or —CH(NHR⁶)—.

16. A process for preparing a compound according to claim 12 of formula Ib: 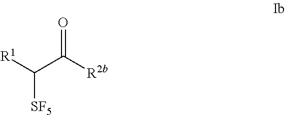
wherein
R¹ is chosen from:
H,
arylalkyl,
alkyl,
alkyl wherein up to three H atoms are replaced with halogen, acyl, alkoxyalkyl, heteroaryl, loweralkoxy, haloalkoxy, aryl, phenoxy, or benzyloxy, and
arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, or benzyloxy; and
R^{2b} is chosen from OH, alkoxy and benzyloxy;
comprising the steps of:
(1) providing a compound of formula Ic: 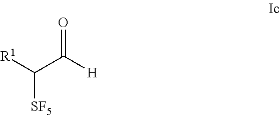
wherein R¹ is as defined above;
(2) oxidizing the compound of formula Ic to a compound of formula Ib wherein R^{2b} is OH; and optionally
(3) esterifying the compound of formula Ib wherein R^{2b} is OH to a compound of formula Ib wherein R^{2b} is alkoxy or benzyloxy.

17. A process according to claim 16 wherein said oxidizing is accomplished using potassium permanganate.

18. A process for preparing a compound according to claim 1 of formula Id: 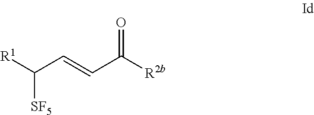
wherein
R¹ is chosen from:
H,
arylalkyl,
alkyl,
alkyl wherein up to three H atoms are replaced with halogen, acyl, alkoxyalkyl, heteroaryl, loweralkoxy, haloalkoxy, aryl, phenoxy, or benzyloxy, and
arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, or benzyloxy; and
R^{2b} is chosen from OH, alkoxy and benzyloxy;
comprising the steps of:
(1) providing a compound of formula Ic: 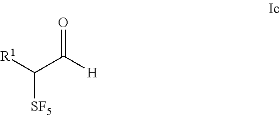
wherein R¹ is as defined above;
(2) converting the compound of formula Ic to a compound of formula Id wherein R^{2b} is alkoxy or benzyloxy; and optionally
(3) converting the compound of formula Id wherein R^{2b} is alkoxy or benzyloxy to a compound of formula Ib wherein R^{2b} is OH.

19. A process according to claim 18 wherein converting said compound of formula Ic to a compound of formula Id is accomplished by treatment of Ic with a phosphonate carbanion or a phosphonium ylide.

20. A process for preparing a compound according to claim 1 of formula Ie: 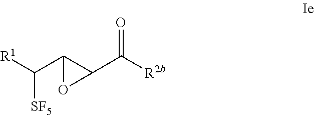
wherein
R¹ is chosen from:
H,
arylalkyl,
alkyl,
alkyl wherein up to three H atoms are replaced with halogen, acyl, alkoxyalkyl, heteroaryl, loweralkoxy, haloalkoxy, aryl, phenoxy, or benzyloxy, and
arylalkyl wherein up to three H atoms on the aryl are replaced with halogen, haloalkyl, alkyl, acyl, alkoxyalkyl, heteroaryl, loweralkoxy, haloalkoxy, cyano, aryl, benzyl, phenoxy, or benzyloxy; and
R^{2b} is chosen from OH, alkoxy and benzyloxy;
comprising the steps of:
(1) providing a compound of formula Ic: 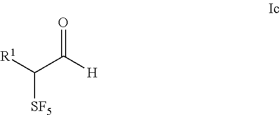
wherein R¹ is as defined above;
(2) converting the compound of formula Ic to a compound of formula Ie wherein R^{2b} is alkoxy or benzyloxy; and optionally
(3) converting the compound of formula Ie wherein R^{2b} is alkoxy or benzyloxy to a compound of formula Ib wherein R^{2b} is OH.

21. A process according to claim 20 wherein converting said compound of formula Ic to a compound of formula Ie is accomplished by treatment of Ic with a sulfonium glide.

22. A process according to claim 15 wherein said converting the compound of formula IIIa to a compound of formula IVa is accomplished by contacting said compound of formula IIIa with a hexane solution of SF₅X.

23. A process according to claim 15 wherein said converting the compound of formula IIa to a compound of formula IIIa is accomplished by
(a) reacting said compound of formula IIa with an ester of R³COOH in the presence of an acid; or
(b) reacting said compound of formula IIa with an anhydride of R³COOH in the presence of a salt of said R³COOH.

24. A process according to claim 23 wherein R³ is phenyl or C₁-C₄ alkyl, in (a) said ester of R³COOH is R³COOCH₂CH₂C(CH₃)═CH₂ and said acid is a sulfonic acid.

25. A process according to claim 23 wherein R³ is phenyl or C₁-C₄ alkyl, in (b) said anhydride of R³COOH is (R³CO)₂O and said salt of R³COOH is an alkali metal salt.
